(19) United States
(12) Patent Application Publication
(10) Pub. No.: US 2025/0136551 A1
(43) Pub. Date: May 1, 2025

(54) DICATIONIC IONIC LIQUID ELECTROLYTES WITH HIGH IONIC CONDUCTIVITY

(71) Applicants: Pradip BHOWMIK, Las Vegas, NV (US); Haesook HAN, Las Vegas, NV (US); Si Lok CHEN, Las Vegas, NV (US)

(72) Inventors: Pradip BHOWMIK, Las Vegas, NV (US); Haesook HAN, Las Vegas, NV (US); Si Lok CHEN, Las Vegas, NV (US)

(21) Appl. No.: 18/835,955

(22) PCT Filed: Feb. 17, 2023
(86) PCT No.: PCT/US2023/013284

§ 371 Date: Aug. 5, 2024

Related U.S. Application Data
(60) Provisional application No. 63/401,522, filed on Aug. 26, 2022.
(60) Provisional application No. 63/313,433, filed on Feb. 24, 2022.
(60) Provisional application No. 63/311,630, filed on Feb. 18, 2022.

(51) Int. Cl. C07D 213/22; H01G 11/62; H01M 10/0568

(52) U.S. Cl. CPC C07D 213/22; H01G 11/62; H01M 10/0568

(57) ABSTRACT

Described herein are non-flammable dicationic ionic liquid electrolytes and the synthesis thereof. The electrolytes exhibit extremely high ionic-conductivities >0.01 S·cm⁻¹. Also described are the use of non-flammable dicationic ionic liquid electrolytes in various energy storage devices such as lithium-ion batteries, rechargeable batteries, fuel cells, super capacitors, or solar cells.

### CROSS-REFERENCE TO RELATED APPLICATIONS

This application claims priority to U.S. Provisional Patent Application Nos. 63/311,630, filed Feb. 18, 2022; 63/313,433, filed Feb. 24, 2022; and 63/401,522, filed Aug. 26, 2022, each of which is incorporated by reference herein in its entirety.

### TECHNICAL FIELD

Described herein are non-flammable dicationic ionic liquid electrolytes and the synthesis thereof. The electrolytes exhibit extremely high ionic-conductivities >0.01 S·cm⁻¹. Also described are the use of non-flammable dicationic ionic liquid electrolytes in various energy storage devices such as lithium-ion batteries, rechargeable batteries, fuel cells, super capacitors, or solar cells.

### BACKGROUND

Energy storage and conversion devices such as lithium-ion batteries, super capacitors, solar cells, fuel cells are gaining wide attention over the past decade. Electrolytes play a crucial role in all these devices. However, flammability, low ionic conductivity and low ion-transference number of existing electrolytes have always been a challenge for these products.

Ionic liquids (ILs) are salts consisting of organic cations and inorganic/organic anions having melting transitions (Tₘ) lower than 100° C. Indeed, many ILs melt well below ambient temperatures, with typical glass transition temperatures (T_{g}) in the range from −93 to −53° C. They hold promise as engineered materials in a variety of modern fields, including green solvents or catalysts for chemical reactions, biocatalysts, biopolymers processing, active pharmaceutical ingredients in medicine, and electrolytes for batteries. Multi-charged ILs and poly(ionic liquid)s exhibit a wider range of physical properties than their mono-charged analogues, e.g., higher density, T_{g}, Tₘ, surface tension and viscosity, due to their higher molecular weights. These result in superior properties, such as, higher thermal stabilities, better antimicrobial activity, higher electrical capacities, better performance as stationary phases for gas chromatography, among others. Multi-charged ILs are particularly attractive due to their combination of low viscosity (like traditional ILs) and high ionic conductivity (like poly(ionic liquid)s). The physical properties of multi-charged ILs can be fine-tuned by combining different cations and anions, with well-defined chemical structures that avoid polydispersity issues. Current multi-charged ILs range ammonium, phosphonium, imidazolium, pyridinium, pyrrolidinium, piperidinium, triazolium and 4,4′-bipyridinium (viologen) cations. The majority of these multi-charged ionic liquids are synthesized via quaternization S_{N}² Menshutkin reactions, followed by metathesis of anions.

What is needed are non-toxic electrolytes that are non-flammable, non-volatile, exhibit high ionic conductivity, high ion-transference number, high electrochemical window, and have high thermal stability.

### SUMMARY

One embodiment described herein is an ionic liquid electrolyte comprising: a viologen salt of formula (I):

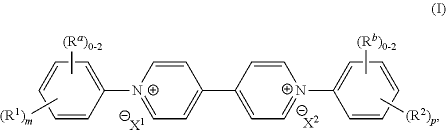

wherein:
R¹ and R² are each independently

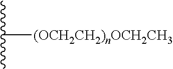

where n is 3-10;
m and p are each independently 1-3;
R^{a}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃;
R^{b}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃; and
^{⊖}X¹ and ^{⊖}X² are each

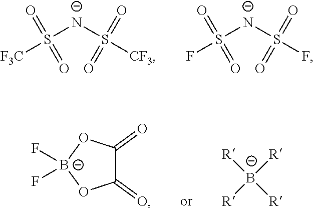

where R′ is —(OCH₂CH₂)₁₋₁₀OCH₂CH₃. In one aspect, m is 1. In another aspect, p is 1. In another aspect, m is 2. In another aspect, p is 2. In another aspect, the viologen salt of formula (I) is a symmetric viologen salt.

In another aspect, ^{⊖}X¹ and ^{⊖}X² are each

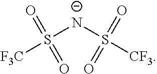 [SMILES: O=S(=O)([N-]S(=O)(=O)C(F)(F)F)C(F)(F)F]

In another aspect, the viologen salt of formula (I) is a viologen salt of formula (I-a):

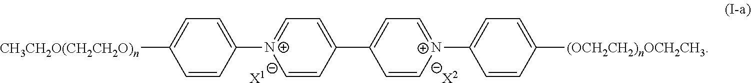

In another aspect, the viologen salt of formula (I) is a viologen salt of formula (I-b):

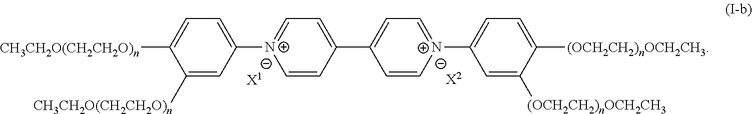

In another aspect, the viologen salt of formula (I) is:

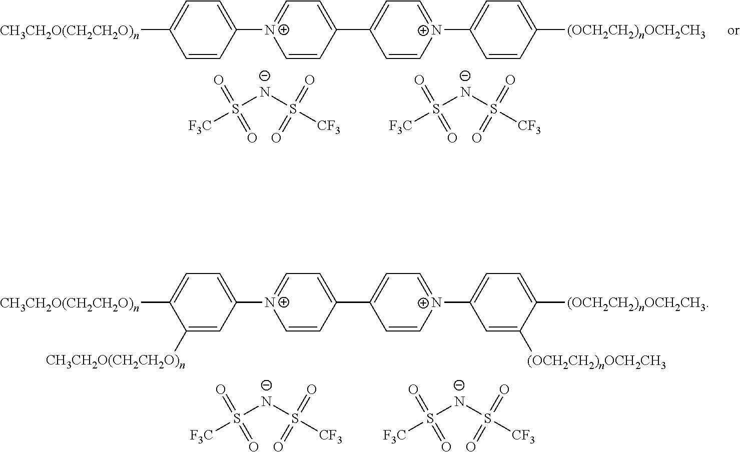

Another embodiment described herein is a solid-state battery, supercapacitor, solar cell, or perovskite photovoltaic cell comprising the ionic liquid electrolyte of claim 1. In another aspect, the viologen salt of formula (I) has a conductivity of greater than 0.1 S·cm⁻¹.

Another embodiment described herein is a solid-state battery comprising a cathode; an anode; a separator; and an ionic liquid electrolyte, wherein the ionic liquid electrolyte comprises: a viologen salt of formula (I):

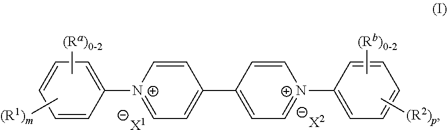

wherein:
R¹ and R² are each independently

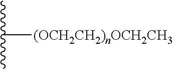

where n is 3-10;
m and p are each independently 1-3;
R^{a}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃;
R^{b}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃; and
^{⊖}X¹ and ^{⊖}X² are each

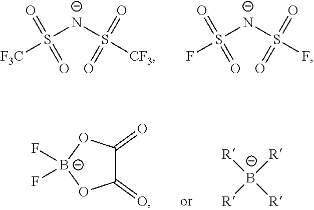

where R′ is —(OCH₂CH₂)₁₋₁₀OCH₂CH₃.

Another embodiment described herein is a supercapacitor comprising: a cathode; an anode; a separator; and an ionic liquid electrolyte, wherein the ionic liquid electrolyte comprises: a viologen salt of formula (I):

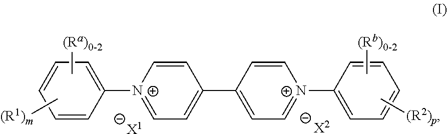

wherein:
R¹ and R² are each independently

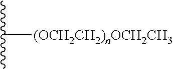

where n is 3-10; m and p are each independently 1-3;
R^{a}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃;
R^{b}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃; and
^{⊖}X¹ and ^{⊖}X² are each

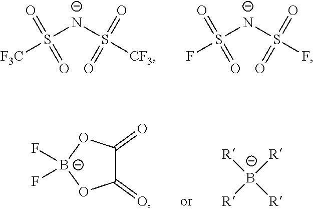

where R′ is —(OCH₂CH₂)₁₋₁₀OCH₂CH₃.

Another embodiment described herein is a solar cell comprising: a n-type semiconductor layer; a p-type semiconductor layer; and an ionic liquid electrolyte, wherein the ionic liquid electrolyte comprises: a viologen salt of formula (I):

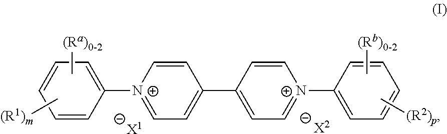

wherein:
R¹ and R² are each independently

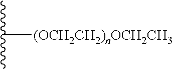

where n is 3-10; m and p are each independently 1-3;
R^{a}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃;
R^{b}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃; and
^{⊖}X¹ and ^{⊖}X² are each

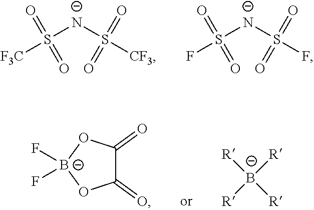

where R′ is —(OCH₂CH₂)₁₋₁₀OCH₂CH₃.

Another embodiment described herein is a perovskite photovoltaic cell comprising: a n-type semiconductor layer; an electron transport layer (ETL); a p-type semiconductor layer; and
an ionic liquid electrolyte, wherein the ionic liquid electrolyte comprises: a viologen salt of formula (I):

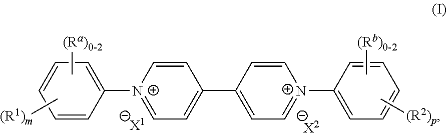

wherein:
R¹ and R² are each independently

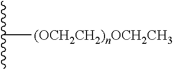

where n is 3-10;
m and p are each independently 1-3;
R^{a}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃;
R^{b}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃; and
^{⊖}X¹ and ^{⊖}X² are each

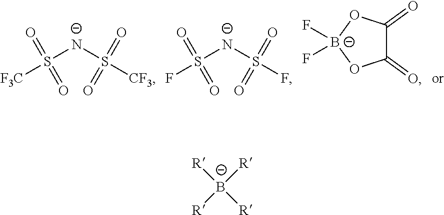

where R′ is —(OCH₂CH₂)₁₋₁₀OCH₂CH₃. In one aspect, m is 1. In another aspect, p is 1. In another aspect, m is 2. In another aspect, p is 2. In another aspect, the viologen salt is a symmetric viologen salt.

In another aspect, ^{⊖}X¹ and ^{⊖}X² are each

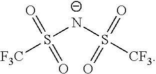 [SMILES: O=S(=O)([N-]S(=O)(=O)C(F)(F)F)C(F)(F)F]

In another aspect, the viologen salt of formula (I) is a viologen salt of formula (I-a):

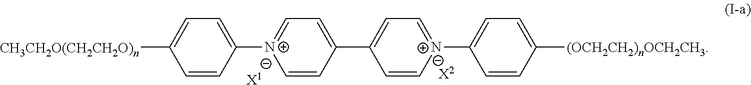

In another aspect, the viologen salt of formula (I) is a viologen salt of formula (I-b):

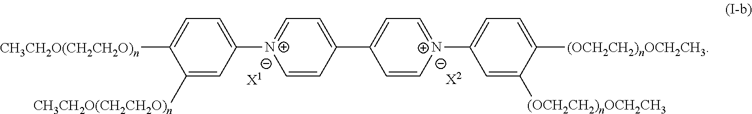

In another aspect, the viologen salt of formula (I) has a conductivity of greater than 0.1 S·cm⁻¹.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-E show different cationic structures containing triflimide anion (Tf)₂N⁻ (FIG. 1A); stilbazolium salts, n=10, 12 (FIG. 1B); asymmetric viologens, n=7, 11, 14 (FIG. 1C); ionic salts prepared in this as described herein, 1 (n=1), 2 (n=2) and 3 (n=3) (FIG. 1D); and alkoxy-terminated viologens (FIG. 1E).

FIG. 2 shows DSC thermograms of 1-3 obtained at a heating rate of 10° C. min⁻¹ in nitrogen. Exo up.

FIG. 3A-C show the dielectric loss modulus, ε″, obtained at isothermal steps for 1 (FIG. 3A), 2 (FIG. 3B), and 3 (FIG. 3C), on heating from room temperature (see arrows).

FIG. 4A-C show the real component of the complex conductivity, σ′, obtained at isothermal steps for 1 (FIG. 4A), 2 (FIG. 4B), and 3 (FIG. 4C), on heating from room temperature (see arrows).

FIG. 5 shows Arrhenius plots (base 10) of 1-3 corresponding to the direct current conductivity, σ_{dc}, estimated from the plateaus in the double logarithmic α′ vs frequency plots.

FIG. 6 shows TGA thermograms of 1, 2, and 3 obtained a heating rate of 10° C./min in nitrogen.

FIG. 7A-C shows DSC thermograms of 11 (FIG. 7A), 2 (FIG. 7B), and 3 (FIG. 7C) obtained at heating and cooling rates of 10° C./min in nitrogen.

FIG. 8 shows a Cole-Cole plot of 3 at 30, 50 and 80° C. The AC oscillation voltage was 0.5 V.

### DETAILED DESCRIPTION

Unless otherwise defined, all technical and scientific terms used herein have the same meaning as commonly understood by one of ordinary skill in the art. For example, any nomenclatures used in connection with, and techniques of synthetic chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkoxy,” as used herein, refers to a group —O-alkyl. Representative examples of alkoxy include, but are not limited to, methoxy, ethoxy, propoxy, 2-propoxy, butoxy and tert-butoxy.

The term “alkyl,” as used herein, means a straight or branched, saturated hydrocarbon chain. The term “lower alkyl” or “C₁₋₆alkyl” means a straight or branched chain hydrocarbon containing from 1 to 6 carbon atoms. The term “C₁₋₄alkyl” means a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl,” as used herein, means a straight or branched, hydrocarbon chain containing at least one carbon-carbon double bond.

The term “alkoxyalkyl,” as used herein, refers to an alkoxy group, as defined herein, appended to the parent molecular moiety through an alkyl group, as defined herein.

The term “alkoxyfluoroalkyl,” as used herein, refers to an alkoxy group, as defined herein, appended to the parent molecular moiety through a fluoroalkyl group, as defined herein.

The term “alkylene,” as used herein, refers to a divalent group derived from a straight or branched chain hydrocarbon of 1 to 10 carbon atoms, for example, of 2 to 5 carbon atoms. Representative examples of alkylene include, but are not limited to, —CH₂—, —CD₂-, —CH₂CH₂—, —CH₂CH₂CH₂—, —CH₂CH₂CH₂CH₂—, and —CH₂CH₂CH₂CH₂CH₂—.

The term “alkylamino,” as used herein, means at least one alkyl group, as defined herein, is appended to the parent molecular moiety through an amino group, as defined herein.

The term “amide,” as used herein, means —C(O)NR— or —NRC(O)—, wherein R may be hydrogen, alkyl, cycloalkyl, aryl, heteroaryl, heterocycle, alkenyl, or heteroalkyl.

The term “aminoalkyl,” as used herein, means at least one amino group, as defined herein, is appended to the parent molecular moiety through an alkylene group, as defined herein.

The term “amino,” as used herein, means —NRₓR_{y}, wherein Rₓ and R_{y} may be hydrogen, alkyl, cycloalkyl, aryl, heteroaryl, heterocycle, alkenyl, or heteroalkyl. In the case of an aminoalkyl group or any other moiety where amino appends together two other moieties, amino may be —NRₓ—, wherein Rₓ may be hydrogen, alkyl, cycloalkyl, aryl, heteroaryl, heterocycle, alkenyl, or heteroalkyl.

The term “aryl,” as used herein, refers to a phenyl or a phenyl appended to the parent molecular moiety and fused to a cycloalkane group (e.g., the aryl may be indan-4-yl), fused to a 6-membered arene group (i.e., the aryl is naphthyl), or fused to a non-aromatic heterocycle (e.g., the aryl may be benzo[d][1,3]dioxol-5-yl). The term “phenyl” is used when referring to a substituent and the term 6-membered arene is used when referring to a fused ring. The 6-membered arene is monocyclic (e.g., benzene or benzo). The aryl may be monocyclic (phenyl) or bicyclic (e.g., a 9- to 12-membered fused bicyclic system).

The term “cyanoalkyl,” as used herein, means at least one —CN group, is appended to the parent molecular moiety through an alkylene group, as defined herein.

The term “cyanofluoroalkyl,” as used herein, means at least one —CN group, is appended to the parent molecular moiety through a fluoroalkyl group, as defined herein.

The term “cycloalkoxy,” as used herein, refers to a cycloalkyl group, as defined herein, appended to the parent molecular moiety through an oxygen atom.

The term “cycloalkyl” or “cycloalkane,” as used herein, refers to a saturated ring system containing all carbon atoms as ring members and zero double bonds. The term “cycloalkyl” is used herein to refer to a cycloalkane when present as a substituent. A cycloalkyl may be a monocyclic cycloalkyl (e.g., cyclopropyl), a fused bicyclic cycloalkyl (e.g., decahydronaphthalenyl), or a bridged cycloalkyl in which two non-adjacent atoms of a ring are linked by an alkylene bridge of 1, 2, 3, or 4 carbon atoms (e.g., bicyclo[2.2.1]heptanyl).

Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, and bicyclo[1.1.1]pentanyl.

The term “cycloalkenyl” or “cycloalkene,” as used herein, means a non-aromatic monocyclic or multicyclic ring system containing all carbon atoms as ring members and at least one carbon-carbon double bond and preferably having from 5-10 carbon atoms per ring. The term “cycloalkenyl” is used herein to refer to a cycloalkene when present as a substituent. A cycloalkenyl may be a monocyclic cycloalkenyl (e.g., cyclopentenyl), a fused bicyclic cycloalkenyl (e.g., octahydronaphthalenyl), or a bridged cycloalkenyl in which two non-adjacent atoms of a ring are linked by an alkylene bridge of 1, 2, 3, or 4 carbon atoms (e.g., bicyclo[2.2.1]heptenyl).

Exemplary monocyclic cycloalkenyl rings include cyclopentenyl, cyclohexenyl or cycloheptenyl. Exemplary monocyclic cycloalkenyl rings include cyclopentenyl, cyclohexenyl or cycloheptenyl.

The term “carbocyclyl” means a “cycloalkyl” or a “cycloalkenyl.” The term “carbocycle” means a “cycloalkane” or a “cycloalkene.” The term “carbocyclyl” refers to a “carbocycle” when present as a substituent.

The terms cycloalkylene and heterocyclylene refer to divalent groups derived from the base ring, i.e., cycloalkane, heterocycle. For purposes of illustration, examples of cycloalkylene and heterocyclylene include, respectively,

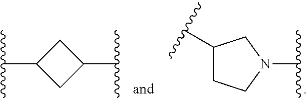 [SMILES: *C1CC(*)C1 ; *C1CCN(*)C1]

Cycloalkylene and heterocyclylene include a geminal divalent groups such as 1,1-C₃₋₆cycloalkylene

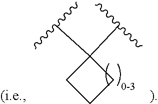

A further example is 1,1-cyclopropylene

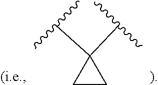 [SMILES: *C1(*)CC1]

The term “fluoroalkyl,” as used herein, means an alkyl group, as defined herein, in which one, two, three, four, five, six, seven or eight hydrogen atoms are replaced by fluorine.

Representative examples of fluoroalkyl include, but are not limited to, 2-fluoroethyl, 2,2,2-trifluoroethyl, trifluoromethyl, difluoromethyl, pentafluoroethyl, and trifluoropropyl such as 3,3,3-trifluoropropyl.

The term “fluoroalkylene,” as used herein, means an alkylene group, as defined herein, in which one, two, three, four, five, six, seven or eight hydrogen atoms are replaced by fluorine.

Representative examples of fluoroalkyl include, but are not limited to —CF₂—, —CH₂CF₂—, 1,2-difluoroethylene, 1,1,2,2-tetrafluoroethylene, 1,3,3,3-tetrafluoropropylene, 1,1,2,3,3-pentafluoropropylene, and perfluoropropylene such as 1,1,2,2,3,3-hexafluoropropylene.

The term “halogen” or “halo,” as used herein, means Cl, Br, I, or F.

The term “haloalkyl,” as used herein, means an alkyl group, as defined herein, in which one, two, three, four, five, six, seven or eight hydrogen atoms are replaced by a halogen.

The term “haloalkoxy,” as used herein, means at least one haloalkyl group, as defined herein, is appended to the parent molecular moiety through an oxygen atom.

The term “halocycloalkyl,” as used herein, means a cycloalkyl group, as defined herein, in which one or more hydrogen atoms are replaced by a halogen.

The term “heteroalkyl,” as used herein, means an alkyl group, as defined herein, in which one or more of the carbon atoms has been replaced by a heteroatom selected from S, O, P and N. Representative examples of heteroalkyls include, but are not limited to, alkyl ethers, secondary and tertiary alkyl amines, amides, and alkyl sulfides.

The term “heteroaryl,” as used herein, refers to an aromatic monocyclic heteroatom-containing ring (monocyclic heteroaryl) or a bicyclic ring system containing at least one monocyclic heteroaromatic ring (bicyclic heteroaryl). The term “heteroaryl” is used herein to refer to a heteroarene when present as a substituent. The monocyclic heteroaryl are five or six membered rings containing at least one heteroatom independently selected from the group consisting of N, O and S (e.g., 1, 2, 3, or 4 heteroatoms independently selected from O, S, and N). The five membered aromatic monocyclic rings have two double bonds, and the six membered aromatic monocyclic rings have three double bonds. The bicyclic heteroaryl is an 8- to 12-membered ring system and includes a fused bicyclic heteroaromatic ring system (i.e., 10π electron system) such as a monocyclic heteroaryl ring fused to a 6-membered arene (e.g., quinolin-4-yl, indol-1-yl), a monocyclic heteroaryl ring fused to a monocyclic heteroarene (e.g., naphthyridinyl), and a phenyl fused to a monocyclic heteroarene (e.g., quinolin-5-yl, indol-4-yl). A bicyclic heteroaryl/heteroarene group includes a 9-membered fused bicyclic heteroaromatic ring system having four double bonds and at least one heteroatom contributing a lone electron pair to a fully aromatic 10π electron system, such as ring systems with a nitrogen atom at the ring junction (e.g., imidazopyridine) or a benzoxadiazolyl. A bicyclic heteroaryl also includes a fused bicyclic ring system composed of one heteroaromatic ring and one non-aromatic ring such as a monocyclic heteroaryl ring fused to a monocyclic carbocyclic ring (e.g., 6,7-dihydro-5H-cyclopenta[b]pyridinyl), or a monocyclic heteroaryl ring fused to a monocyclic heterocycle (e.g., 2,3-dihydrofuro[3,2-b]pyridinyl). The bicyclic heteroaryl is attached to the parent molecular moiety at an aromatic ring atom. Other representative examples of heteroaryl include, but are not limited to, indolyl (e.g., indol-1-yl, indol-2-yl, indol-4-yl), pyridinyl (including pyridin-2-yl, pyridin-3-yl, pyridin-4-yl), pyrimidinyl, pyrazinyl, pyridazinyl, pyrazolyl (e.g., pyrazol-4-yl), pyrrolyl, benzopyrazolyl, 1,2,3-triazolyl (e.g., triazol-4-yl), 1,3,4-thiadiazolyl, 1,2,4-thiadiazolyl, 1,3,4-oxadiazolyl, 1,2,4-oxadiazolyl, imidazolyl, thiazolyl (e.g., thiazol-4-yl), isothiazolyl, thienyl, benzimidazolyl (e.g., benzimidazol-5-yl), benzothiazolyl, benzoxazolyl, benzoxadiazolyl, benzothienyl, benzofuranyl, isobenzofuranyl, furanyl, oxazolyl, isoxazolyl, purinyl, isoindolyl, quinoxalinyl, indazolyl (e.g., indazol-4-yl, indazol-5-yl), quinazolinyl, 1,2,4-triazinyl, 1,3,5-triazinyl, isoquinolinyl, quinolinyl, imidazo[1,2-a]pyridinyl (e.g., imidazo[1,2-a]pyridin-6-yl), naphthyridinyl, pyridoimidazolyl, thiazolo[5,4-b]pyridin-2-yl, and thiazolo[5,4-d]pyrimidin-2-yl.

The term “heterocycle” or “heterocyclic,” as used herein, means a monocyclic heterocycle, a bicyclic heterocycle, or a tricyclic heterocycle. The term “heterocyclyl” is used herein to refer to a heterocycle when present as a substituent. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocyclyls include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, 2-oxo-3-piperidinyl, 2-oxoazepan-3-yl, oxadiazolinyl, oxadiazolidinyl, oxazolinyl, oxazolidinyl, oxetanyl, oxepanyl, oxocanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a 6-membered arene, or a monocyclic heterocycle fused to a monocyclic cycloalkane, or a monocyclic heterocycle fused to a monocyclic cycloalkene, or a monocyclic heterocycle fused to a monocyclic heterocycle, or a monocyclic heterocycle fused to a monocyclic heteroarene, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. The bicyclic heterocyclyl is attached to the parent molecular moiety at a non-aromatic ring atom (e.g., indolin-1-yl). Representative examples of bicyclic heterocyclyls include, but are not limited to, chroman-4-yl, 2,3-dihydrobenzofuran-2-yl, 2,3-dihydrobenzothien-2-yl, 1,2,3,4-tetrahydroisoquinolin-2-yl, 2-azaspiro[3.3]heptan-2-yl, 2-oxa-6-azaspiro[3.3]heptan-6-yl, azabicyclo[2.2.1]heptyl (including 2-azabicyclo[2.2.1]hept-2-yl), azabicyclo[3.1.0]hexanyl (including 3-azabicyclo[3.1.0]hexan-3-yl), 2,3-dihydro-1H-indol-1-yl, isoindolin-2-yl, octahydrocyclopenta[c]pyrrolyl, octahydropyrrolopyridinyl, tetrahydroisoquinolinyl, 7-oxabicyclo[2.2.1]heptanyl, hexahydro-2H-cyclopenta[b]furanyl, 2-oxaspiro[3.3]heptanyl, 3-oxaspiro[5.5]undecanyl, 6-oxaspiro[2.5]octan-1-yl, and 3-oxabicyclo[3.1.0]hexan-6-yl. Tricyclic heterocycles are exemplified by a bicyclic heterocycle fused to a 6-membered arene, or a bicyclic heterocycle fused to a monocyclic cycloalkane, or a bicyclic heterocycle fused to a monocyclic cycloalkene, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.13,7]decane), and oxa-adamantane (2-oxatricyclo[3.3.1.13,7]decane). The monocyclic, bicyclic, and tricyclic heterocyclyls are connected to the parent molecular moiety at a non-aromatic ring atom.

The term “hydroxyl” or “hydroxy,” as used herein, means an —OH group.

The term “hydroxyalkyl,” as used herein, means at least one —OH group, is appended to the parent molecular moiety through an alkylene group, as defined herein.

The term “hydroxyfluoroalkyl,” as used herein, means at least one —OH group, is appended to the parent molecular moiety through a fluoroalkyl group, as defined herein.

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

The term “substituted” refers to a group that may be further substituted with one or more non-hydrogen substituent groups. Substituent groups include, but are not limited to, halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

Described herein are non-flammable dicationic ionic liquid electrolyte exhibiting high ionic conductivity (>0.01 S·cm⁻¹), high ion transference number (>0.5), wide electrochemical window (>6 V) for use in electrical energy storage and conversion devices such as lithium-ion batteries, fuel cell, solar cells, super capacitor. Existing electrolytes based on both conventional organic liquids and ionic liquids all exhibit ionic conductivity in the range of 10⁻⁴-10⁻² S·cm⁻¹ and are highly flammable.

The electrolytes described herein exhibit high ionic conductivity and are non-flammable.

In some embodiments, the ionic liquids described herein can also be used as green solvents or catalysts for chemical reactions, biocatalysts, biopolymers processing, active pharmaceutical ingredients in medicine.

In some embodiments, dicationic ionic liquid electrolytes are achieved by employing a combination of suitable organic cationic and organic/inorganic anionic salts (such as triflimide viologens), along with extended core structures containing oxyethylene(s) terminations.

In some embodiments, these multi-charged ionic liquids and polyionic gels can be synthesized via Zincke reaction.

Embodiments described herein exhibit the following properties: high ionic conductivity (>0.01 S·cm⁻¹); high ion transference number (>0.5); non-flammability; high thermal stability;
non-toxicity; sustainability; liquid in nature below 100° C.; high electrochemical stability window and non-LC (liquid crystalline) nature.

In some embodiments, suitable ammonium, phosphonium, imidazolium, pyridinium, pyrrolidinium, piperidinium, triazolium, 4,4′-bipyridinium and alkoxy-terminated viologen cations may be used.

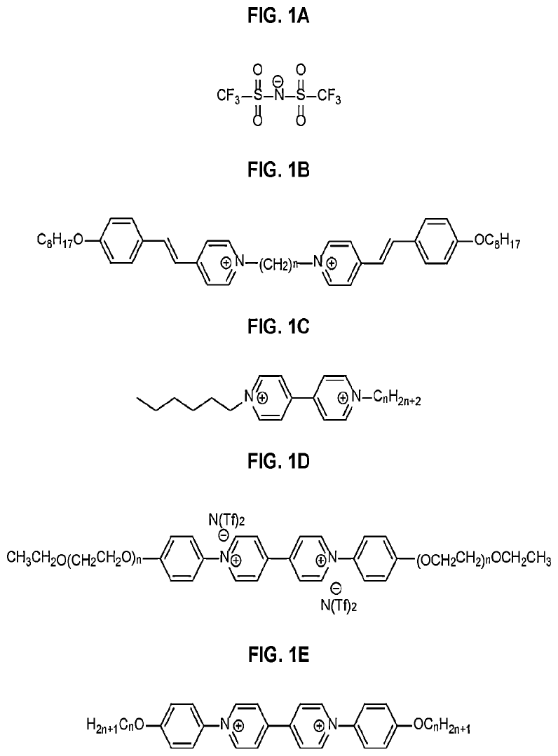

In some embodiments, extended oxyethylene(s) terminations (with n varying from 1 to 15; FIG. 1) may be used.

In some embodiments, the ionic liquids can have melt transition temperatures lower than 100° C. and glass transition temperatures between −93 and −53° C.

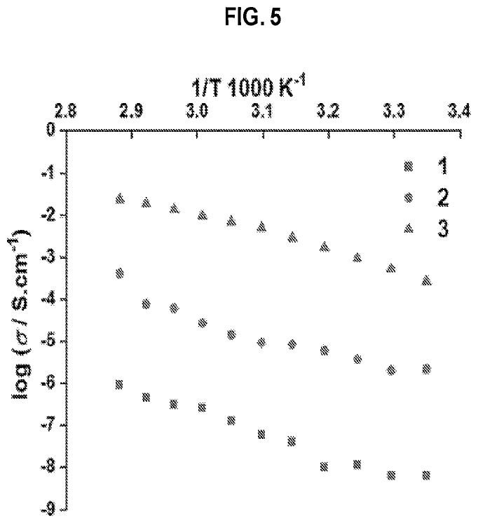

In some embodiments, these ionic liquids will exhibit high thermal stability (see FIG. 5).

In some embodiments, these ionic liquids are amorphous in nature and inhibit crystallization.

In some embodiments, the conductivity is increased by increasing the length of oxyethylene terminals

In some embodiments, these ionic liquids may exhibit conductivity that is weak temperature dependence

In some embodiments, the longer oxyethylene terminals act as plasticizers

In some embodiments, the longer oxyethylene terminals promote ionic motion there by their conductivity

In some embodiments, these ionic liquids can exhibit high electrochemical stability window (>6 V)

One embodiment described herein is an ionic liquid electrolyte comprising: a viologen salt of formula (I):

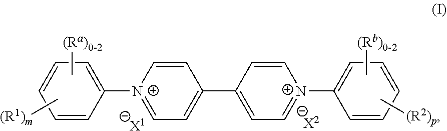

wherein:
R¹ and R² are each independently

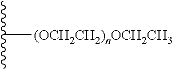

where n is 3-10;
m and p are each independently 1-3;
R^{a}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃;
R^{b}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃; and
^{⊖}X¹ and ^{⊖}X² are each

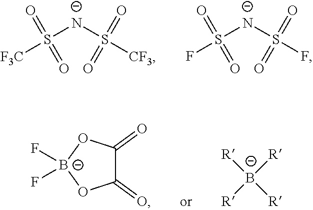

where R′ is —(OCH₂CH₂)₁₋₁₀OCH₂CH₃. In one aspect, m is 1. In another aspect, p is 1. In another aspect, m is 2. In another aspect, p is 2. In another aspect, the viologen salt of formula (I) is a symmetric viologen salt.

In another aspect, ^{⊖}X¹ and ^{⊖}X² are each

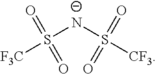 [SMILES: O=S(=O)([N-]S(=O)(=O)C(F)(F)F)C(F)(F)F]

In another aspect, the viologen salt of formula (I) is a viologen salt of formula (I-a):

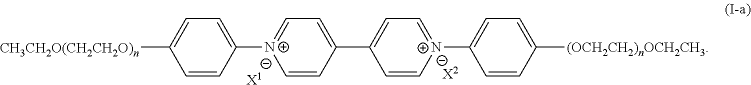

In another aspect, the viologen salt of formula (I) is a viologen salt of formula (I-b):

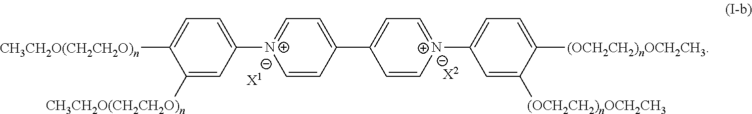

In another aspect, the viologen salt of formula (I) is:

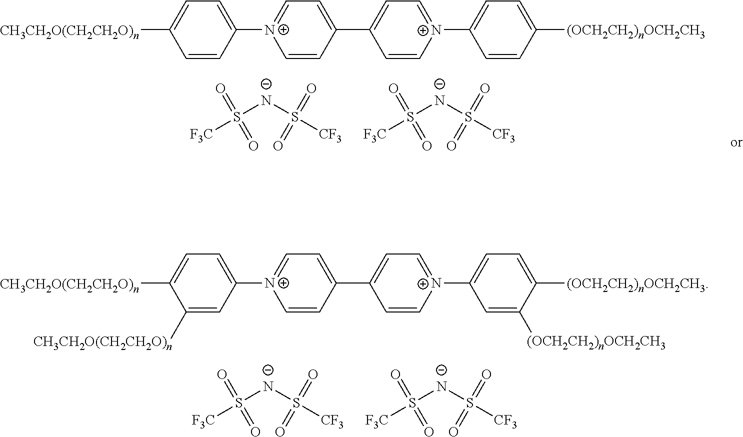

Another embodiment described herein is a solid-state battery, supercapacitor, solar cell, or perovskite photovoltaic cell comprising the ionic liquid electrolyte of claim 1. In another aspect, the viologen salt of formula (I) has a conductivity of greater than 0.1 S·cm⁻¹.

Another embodiment described herein is a solid-state battery comprising a cathode; an anode; a separator; and an ionic liquid electrolyte, wherein the ionic liquid electrolyte comprises: a viologen salt of formula (I):

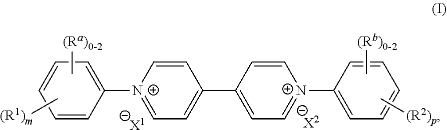

wherein:
R¹ and R² are each independently

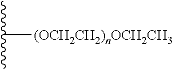

where n is 3-10;
m and p are each independently 1-3;
R^{a}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃;
R^{b}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃; and
^{⊖}X¹ and ^{⊖}X² are each

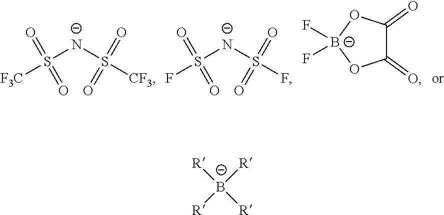

where R′ is —(OCH₂CH₂)₁₋₁₀OCH₂CH₃.

Another embodiment described herein is a supercapacitor comprising: a cathode; an anode; a separator; and an ionic liquid electrolyte, wherein the ionic liquid electrolyte comprises: a viologen salt of formula (I):

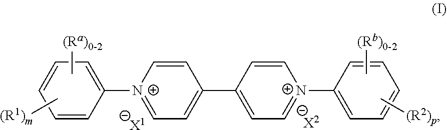

wherein:
R¹ and R² are each independently

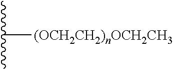

where n is 3-10; m and p are each independently 1-3;
R^{a}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃;
R^{b}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃; and
^{⊖}X¹ and ^{⊖}X² are each

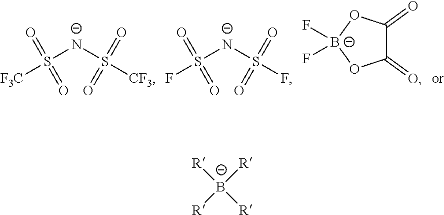

where R′ is —(OCH₂CH₂)₁₋₁₀OCH₂CH₃.

Another embodiment described herein is a solar cell comprising: a n-type semiconductor layer; a p-type semiconductor layer; and an ionic liquid electrolyte, wherein the ionic liquid electrolyte comprises: a viologen salt of formula (I):

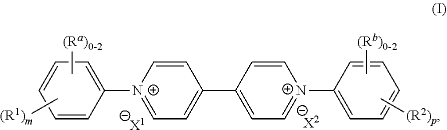

wherein:
R¹ and R² are each independently

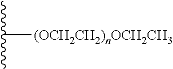

where n is 3-10; m and p are each independently 1-3;
R^{a}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃;
R^{b}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃; and
^{⊖}X¹ and ^{⊖}X² are each

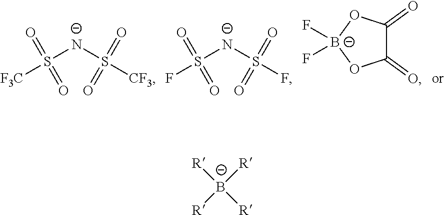

where R′ is —(OCH₂CH₂)₁₋₁₀OCH₂CH₃.

Another embodiment described herein is a perovskite photovoltaic cell comprising: a n-type semiconductor layer; an electron transport layer (ETL); a p-type semiconductor layer; and an ionic liquid electrolyte, wherein the ionic liquid electrolyte comprises: a viologen salt of formula (I):

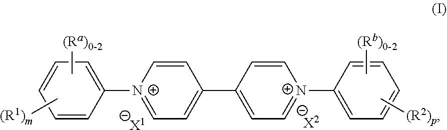

wherein:
R¹ and R² are each independently

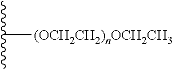

where n is 3-10;
m and p are each independently 1-3;
R^{a}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃;
R^{b}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃; and
^{⊖}X¹ and ^{⊖}X² are each

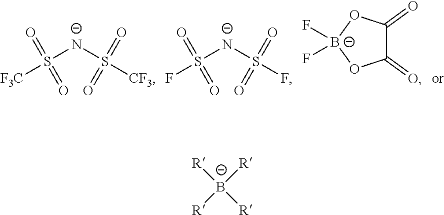

where R′ is —(OCH₂CH₂)₁₋₁₀OCH₂CH₃. In one aspect, m is 1. In another aspect, p is 1. In another aspect, m is 2. In another aspect, p is 2. In another aspect, the viologen salt is a symmetric viologen salt.

In another aspect, ^{⊖}X¹ and ^{⊖}X² are each

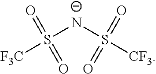 [SMILES: O=S(=O)([N-]S(=O)(=O)C(F)(F)F)C(F)(F)F]

In another aspect, the viologen salt of formula (I) is a viologen salt of formula (I-a):

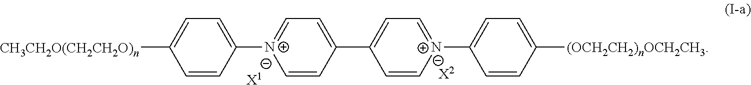

In another aspect, the viologen salt of formula (I) is a viologen salt of formula (I-b):

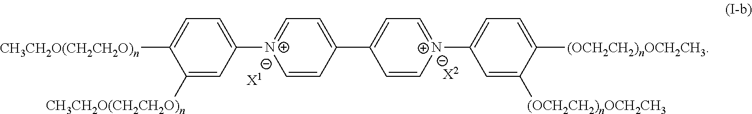

In another aspect, the viologen salt of formula (I) has a conductivity of greater than 0.1 S·cm⁻¹.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:

Clause 1. An ionic liquid electrolyte comprising:
a viologen salt of formula (I):

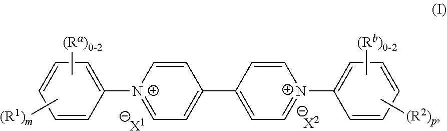

wherein:
R¹ and R² are each independently

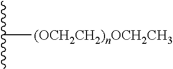

where n is 3-10;
m and p are each independently 1-3;
R^{a}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃;
R^{b}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃; and
^{⊖}X¹ and ^{⊖}X² are each

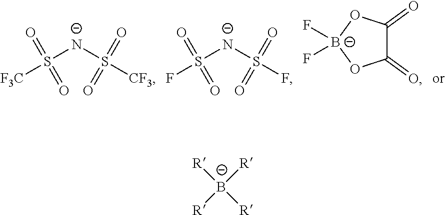

where R′ is —(OCH₂CH₂)₁₋₁₀OCH₂CH₃.

Clause 2. The ionic liquid electrolyte of clause 1, wherein m is 1.

Clause 3. The ionic liquid electrolyte of clause 1, wherein p is 1.

Clause 4. The ionic liquid electrolyte of clause 1, wherein m is 2.

Clause 5. The ionic liquid electrolyte of clause 1, wherein p is 2.

Clause 6. The ionic liquid electrolyte of clause 1, wherein the viologen salt of formula (I) is a symmetric viologen salt.

Clause 7. The ionic liquid electrolyte of clause 1, wherein ^{⊖}X¹ and ^{⊖}X² are each

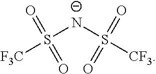 [SMILES: O=S(=O)([N-]S(=O)(=O)C(F)(F)F)C(F)(F)F]

Clause 8. The ionic liquid electrolyte of clause 1, wherein the viologen salt of formula (I) is a viologen salt of formula (I-a):

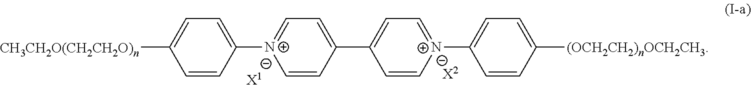

Clause 9. The ionic liquid electrolyte of clause 1, wherein the viologen salt of formula (I) is a viologen salt of formula (I-b):

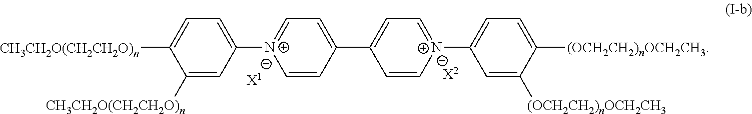

Clause 10. The ionic liquid electrolyte of clause 1, wherein the viologen salt of formula (I) is:

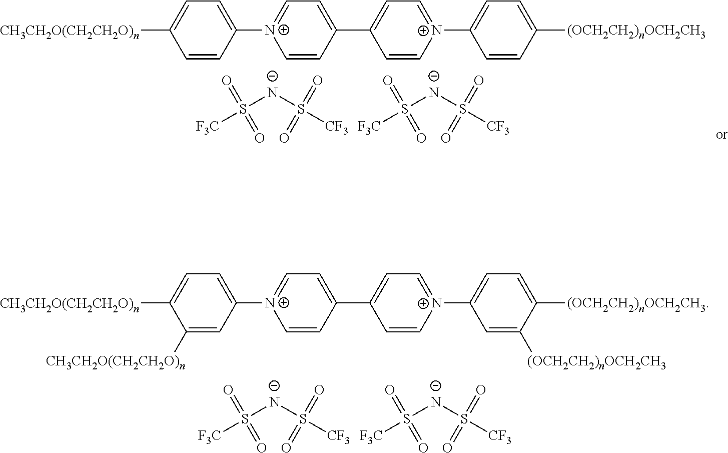

Clause 11. A solid-state battery, supercapacitor, solar cell, or perovskite photovoltaic cell comprising the ionic liquid electrolyte of clause 1.

Clause 12. The ionic liquid electrolyte of clause 1, wherein the viologen salt of formula (I) has a conductivity of greater than 0.1 S·cm⁻¹.

Clause 13. A solid-state battery comprising a cathode;
an anode;
a separator; and
an ionic liquid electrolyte, wherein the ionic liquid electrolyte comprises:
a viologen salt of formula (I):

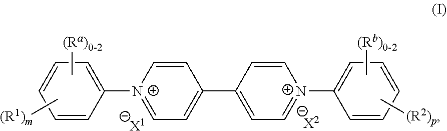

wherein:
R¹ and R² are each independently

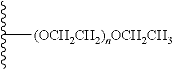

where n is 3-10;
m and p are each independently 1-3;
R^{a}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃;
R^{b}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃; and
^{⊖}X¹ and ^{⊖}X² are each

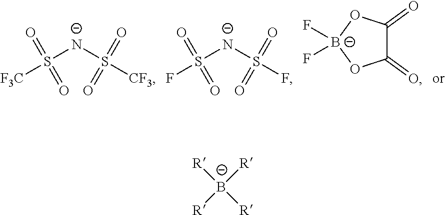

where R′ is —(OCH₂CH₂)₁₋₁₀OCH₂CH₃.

Clause 14. A supercapacitor comprising:
a cathode;
an anode;
a separator; and
an ionic liquid electrolyte, wherein the ionic liquid electrolyte comprises: a viologen salt of formula (I):

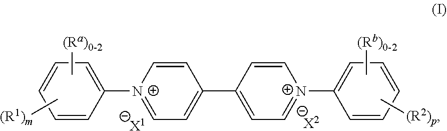

wherein:
R¹ and R² are each independently

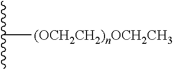

where n is 3-10;
m and p are each independently 1-3;
R^{a}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃;
R^{b}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃; and
^{⊖}X¹ and ^{⊖}X² are each

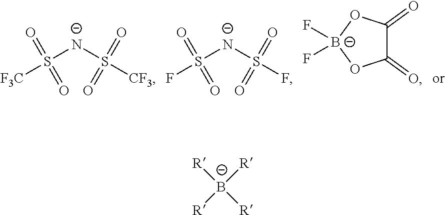

where R′ is —(OCH₂CH₂)₁₋₁₀OCH₂CH₃.

Clause 15. A solar cell comprising:
a n-type semiconductor layer;
a p-type semiconductor layer; and
an ionic liquid electrolyte, wherein the ionic liquid electrolyte comprises: a viologen salt of formula (I):

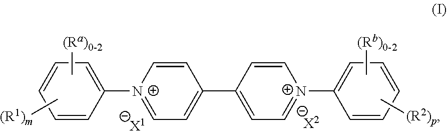

wherein:
R¹ and R² are each independently

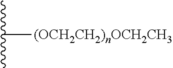

where n is 3-10;
m and p are each independently 1-3;
R^{a}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃;
R^{b}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃; and
^{⊖}X¹ and ^{⊖}X² are each

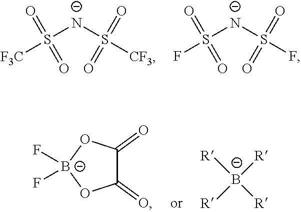

where R′ is —(OCH₂CH₂)₁₋₁₀OCH₂CH₃.

Clause 16. A perovskite photovoltaic cell comprising:
a n-type semiconductor layer;
an electron transport layer (ETL);
a p-type semiconductor layer; and
an ionic liquid electrolyte, wherein the ionic liquid electrolyte comprises: a viologen salt of formula (I):

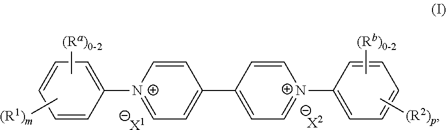

wherein:
R¹ and R² are each independently

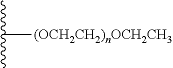

where n is 3-10;
m and p are each independently 1-3;
R^{a}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃;
R^{b}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃; and
^{⊖}X¹ and ^{⊖}X² are each

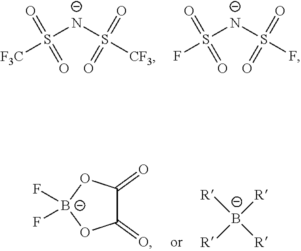

where R′ is —(OCH₂CH₂)₁₋₁₀OCH₂CH₃.

Clause 17. The solid-state battery, supercapacitor, solar cell, or perovskite photovoltaic cell of any one of clauses 13-16, wherein m is 1.

Clause 18. The solid-state battery, supercapacitor, solar cell, or perovskite photovoltaic cell of any one of clauses 13-16, wherein p is 1.

Clause 19. The solid-state battery, supercapacitor, solar cell, or perovskite photovoltaic cell of any one of clauses 13-16, wherein m is 2.

Clause 20. The solid-state battery, supercapacitor, solar cell, or perovskite photovoltaic cell of any one of clauses 13-16, wherein p is 2.

Clause 21. The solid-state battery, supercapacitor, solar cell, or perovskite photovoltaic cell of any one of clauses 13-16, wherein the viologen salt is a symmetric viologen salt.

Clause 22. The solid-state battery, supercapacitor, solar cell, or perovskite photovoltaic cell of any one of clauses 13-16, wherein ^{⊖}X¹ and ^{⊖}X² are each

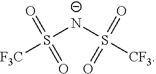 [SMILES: O=S(=O)([N-]S(=O)(=O)C(F)(F)F)C(F)(F)F]

Clause 23. The solid-state battery, supercapacitor, solar cell, or perovskite photovoltaic cell of any one of clauses 13-16, wherein the viologen salt of formula (I) is a viologen salt of formula (I-a):

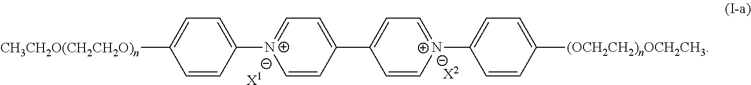

Clause 24. The solid-state battery, supercapacitor, solar cell, or perovskite photovoltaic cell of any one of clauses 13-16, wherein the viologen salt of formula (I) is a viologen salt of formula (I-b):

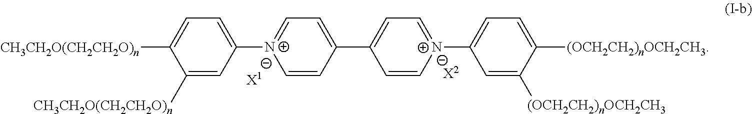

Clause 25. The solid-state battery, supercapacitor, solar cell, or perovskite photovoltaic cell of any one of clauses 13-16, wherein the viologen salt of formula (I) has a conductivity of greater than 0.1 S·cm⁻¹.

### EXAMPLES

### Materials and Methods

All chemicals and solvents were reagent grade and purchased from commercial vendors (Acros Organics, Alfa-Aesar, Sigma-Aldrich, and TCI America) and were used as received. The FTIR spectra were recorded with a Shimadzu infrared spectrometer. The salt samples were prepared in thin films casting from chloroform on NaCl plates and subsequently vacuum dried at 70° C. overnight.

The ¹H, ¹³C and ¹⁹F nuclear magnetic resonance (NMR) sample solutions of extended viologen salts 1-3 were prepared by dissolving 10 mg of each of the salts in 1 mL CD₃OD, and the spectra were recorded by using VNMR 400 spectrometer operating at 400, 100, 376 MHz, respectively, at room temperature and chemical shifts were referenced to tetramethylsilane (TMS) for ¹H and ¹³C nuclei and trichlorofluoromethane (CFCl₃) for ¹⁹F nuclei, respectively. Elemental analyses were performed by Atlantic Microlab Inc., Norcross, GA.

The phase transition temperatures of the compounds were conducted on TA module differential scanning calorimetry DSC Q200 series in nitrogen at heating and cooling rates of 10° C.·min⁻¹. The temperature axis of the DSC thermograms was calibrated with reference standards of high purity indium and tin. The thermal stability properties of the compounds were conducted using a thermogravimetric analysis (TGA) Q50 instrument at a heating rate of 10° C.·min⁻¹ in nitrogen. Polarizing optical microscopy studies of the salts were made by sandwiching them between standard glass coverslips. The samples were heated and cooled on a Mettler hot-stage (FP82HT) and (FP90) controller and had their phase transitions observed between cross polarizers of an Olympus BX51 microscope.

For the dielectric and conductivity analyses, the triflimide salts were sandwiched between two stainless steel electrodes (top and bottom electrodes with diameters of 10 and 20 mm, respectively), separated by a 120 μm-diameter fused silica fiber spacer. The temperature of the sample was controlled using a Lakeshore temperature controller. The dielectric measurements were carried out using a laboratory-built dielectric spectrometer with a frequency range of 0.01-106 Hz. The temperature was measured at 5° C. intervals between −50 and 40° C. At each measurement, the samples were allowed to stabilize at the specified temperature for around two minutes.

Synthetic procedure for 4-oligoethylenoxy anilines

The 4-(2-ethoxyethoxy) aniline and 4-(2-(2-ethoxyethoxy) ethoxy] aniline and 4-(2-(2-(2-ethoxyethoxy) ethoxy)ethoxy]ethoxy] aniline were prepared according to the slightly modified procedure of Sudhakar et al., Liq. Cryst. 27 1525-1532 (2000). The modification was the use of acetone instead of ethanol and of oligoethylene bromides instead of oligoethylene tosylates in the alkylation of 4-hydroxyacetanilide. The oligoethylene bromides were prepared via Appel reaction (Step 1). Sudhakar et al., Liq. Cryst. 27 1525-1532 (2000). The synthesis of 4-[2-[2-(2-ethoxyethoxy) ethoxy)ethoxy]ethoxy] aniline is described below in detail; the other two 4-oligoethyleneoxy anilines were prepared in the identical manner. It was prepared in a three-step reaction starting with the bromination of 4-[2-(2-ethoxyethoxy) ethoxy]ethanol via Appel reaction. Kim et al., J. Med. Chem. 50: 5217-5226 (2007). The synthesis of 4-[2-(2-ethoxyethoxy) ethoxy] bromide is as follows. An amount of 4-[2-(2-ethoxyethoxy) ethoxy] ethanol (1.000 g, 5.61 mmol) and an excess of triphenylphosphine (2.458 g, 9.37 mmol) were added to an Erlenmeyer flask and dissolved in 10 mL of dichloromethane (DCM). When the mixture was stirred and cooled to 0° C., carbon tetrabromide (2.326 g, 7.01 mmol) dissolved in 10 mL of DCM was added to the mixture dropwise. Once all the carbon tetrabromide solution was added, the reaction mixture was let stir at room temperature for 30 min. After 30 min., the DCM was removed using a rotary evaporator. Upon removal of the DCM, 30 mL of hexane was added to the reaction mixture to precipitate out the excess starting material and byproducts. The reaction mixture in hexane was cooled down to −77° C. by keeping the flask in an isopropyl alcohol and dry ice bath. The contents of the flask were filtered through Celite, and the hexane was evaporated leaving a pure product of 4-[2-(2-ethoxyethoxy) ethoxy] ethyl bromide (0.700 g, 2.90 mmol, Yield=52%).

In Step 2, the alkylation of 4-hydroxyacetanilide was performed as follows. 4-[2-(2-ethoxyethoxy) ethoxy] bromide (0.700 g, 2.90 mmol) was added to a round-bottomed flask containing 4-hydroxyacetanilide (0.483 g, 3.19 mmol) dissolved in 50 mL of acetone. Potassium carbonate (0.401 mg, 2.90 mmol) was added to the flask and the reaction mixture was heated to reflux on stirring for 24 h. At the end of the reaction, the mixture was brought to room temperature and filtered. The acetone was removed using a rotary evaporator and the product was purified by extraction with DCM and warm deionized water. The DCM was then evaporated to yield a pure product of 4-[2-[2-(2-ethoxyethoxy) ethoxy] ethoxy] acetanilide (0.800 g, 2.57 mmol, Yield=88%).

Finally, a hydrolysis reaction (Step 2) was performed by adding 4-[2-[2-(2-ethoxyethoxy) ethoxy] ethoxy] acetanilide (0.800 g, 2.57 mmol) to a three-necked flask with sodium hydroxide (2.000 g, 50.0 mmol) dissolved in 25 mL of deionized water. The reaction flask was heated under nitrogen atmosphere for 12 h. At the end of the reaction, the flask was cooled down to room temperature and the desired product was purified by extraction with DCM and deionized water. The DCM was removed using a rotary evaporator to yield a pure product of 4-[2-[2-(2-ethoxyethoxy) ethoxy] ethoxy] aniline (0.576 g, 2.14 mmol, Yield=83%).

### Synthetic Procedure for Zincke Salt.

It was prepared, according to the literature, from the reaction of 1-chloro-2,4-dinitrobenzene (2.5 equivalents) with 4,4′-bipyridine (1 equiv.) on heating in acetonitrile (Step 3) Sharma et al., Synth. Met. 106: 97-105 (1999); Cheng and Kurth, Org. Prep. Proced. Int. 34: 585-608 (2002).

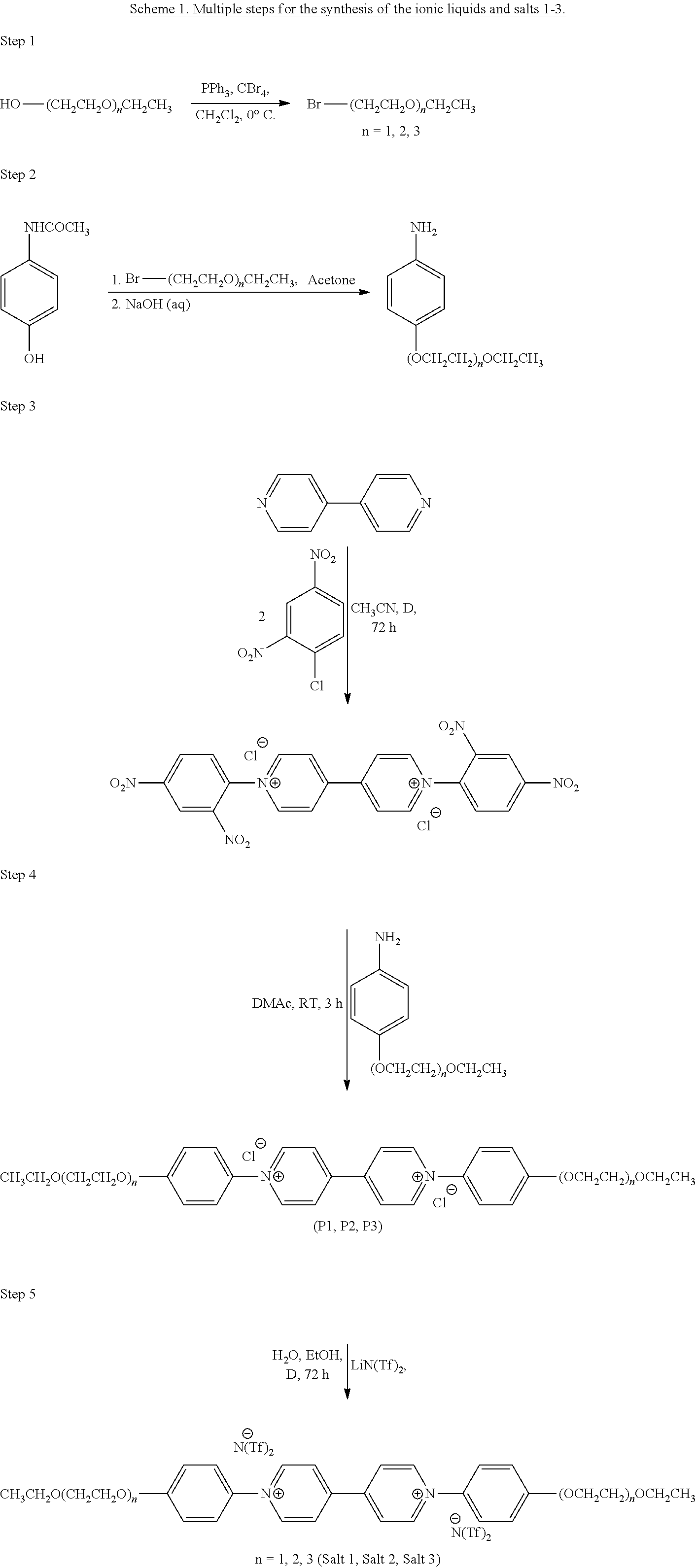

### Synthetic procedure for bis-(4-oligoethyleneoxyphenyl)-4,4′-bipyridinium dichloride (P1-P3)

The synthesis of P2 is described as an example (Step 4); P1 and P3 were prepared in an identical manner. It was prepared by adding the 4-(2-(2-ethoxyethoxy) ethoxy] aniline (0.192 g, 0.85 mmol) to a round-bottomed flask containing Zincke salt (0.217 g, 0.39 mmol) and 15 mL of N,N-dimethylacetamide (DMAc). The reaction mixture was stirred at room temperature for 3 h. At the end of the reaction, the crude product was collected by simply gravity filtration and washed with acetone to give a pure product (0.162 g, 0.25 mmol).

Data for P1: Yield 93%. δH (CD₃OD, 400 MHz, ppm): 9.50 (4H, d, J=6.8), 8.89 (4H, d, J=6.8), 7.89 (4H, d, J=9.2), 7.34 (4H, d, J=9.2), 4.30 (4H, t, J=4.4), 3.87 (4H, t, J=2.8), 3.65 (4H, t, J=6.8) 1.25 (6H, t, J=7.2). δC (CD₃OD, 100 MHz, ppm): 163.18, 151.17, 146.82, 137.02, 128.30, 126.94, 117.44, 69.95, 69.53, 67.85, 15.47.

Data for P2: Yield 65%. δH (CD₃OD, 400 MHz, ppm): 9.50 (4H, br), 8.88 (4H, br), 7.88 (4H, d, J=8.4), 7.34 (4H, d, J=8.8), 4.31 (4H, t, J=4.4), 3.92 (4H, t, J=2.8), 3.73 (4H, t, J=4.4), 3.64 (4H, t, J=2.8), 3.58 (4H, t, J=6.8), 1.21 (6H, t, J=6.8). δC (CD₃OD, 100 MHz, ppm): 163.19, 151.18, 146.83, 137.00, 128.28, 126.93, 117.46, 71.85, 70.95, 70.62, 69.53, 67.64, 15.47.

Data for P3: Yield 70%. δH (CD₃OD, 400 MHz, ppm): 9.50 (4H, d, J=6.4), 8.88 (4H, d, J=6.4), 7.88 (4H, d, J=8.8), 7.34 (4H, d, J=9.2), 4.31 (4H, t, J=4.8), 3.93 (4H, t, J=3.2), 3.75 (4H, t, J=4.0), 3.51-3.69 (16H, m), 1.20 (6H, t, J=6.8). δC (CD₃OD, 100 MHz, ppm): 163.19, 151.18, 146.83, 137.01, 128.28, 126.94, 117.46, 71.84, 71.64, 70.94, 70.62, 69.55, 67.59, 15.48.

### Synthetic procedure for bis-(4-oligoethyleneoxyphenyl)-4,4′-bipyridinium bis(triflimide) 1-3 by metathesis reaction

The salts 1-3 were synthesized from the metathesis reaction of the dichloride salts with lithium triflimide. The synthesis of 2 is described as an example (Step 5); 1 and 3 were prepared in an identical manner. The lithium salt (0.316 g, 1.10 mmol) dissolved in 5 mL of deionized water was added to a reaction flask containing a clear solution of P2 (0.284 g, 0.44 mmol) dissolved in 20 mL of ethanol. The flask was heated to reflux for 72 h. At the end of the reaction, the solvent ethanol was removed by using a rotary evaporator. The reaction mixture was then dissolved in chloroform and extracted from deionized water to give a pure brown product (0.466 g, 0.41 mmol).

Data for 1: FTIR (NaCl, νₘₐₓ cm⁻¹): 3117, 3071, 2978, 2932, 2870, 1628, 1597, 1450, 1342, 1180, 1126, 1049, 826, 787, 733, 610, 571, 501. Yield 70%. δH (CD₃OD, 400 MHz, ppm): 9.46 (4H, br), 8.81 (4H, br), 7.84 (4H, d, J=8.4), 7.33 (4H, d, J=9.2), 4.29 (4H, t, J=4.8), 3.87 (4H, t, J=2.8), 3.65 (4H, t, J=7.2) 1.25 (6H, t, J=7.2). δC (CD₃OD, 100 MHz, ppm): 163.20, 146.83, 128.27, 126.91, 122.77, 119.58, 117.44, 69.95, 69.52, 67.86, 15.46. δF (CD₃OD, 376 MHz, ppm): −80.14. Anal. Calc for C₃₄H₃₄F₁₂N₄O₁₂S₄ (1046.89): C, 39.01; H, 3.27; N, 5.35; S, 12.25%. Found C, 39.17; H, 3.19; N, 5.46; S, 12.25%.

Data for 2: FTIR (NaCl, νₘₐₓ cm⁻¹): 3125, 3071, 2978, 2932, 2878, 1636, 1597, 1543, 1504, 1450, 1427, 1350, 1258, 1196, 1134, 1096, 1057, 941, 833, 795, 741, 610, 571, 509. Yield 93%. δH (CD₃OD, 400 MHz, ppm): 9.47 (4H, d, J=5.6), 8.82 (4H, d, J=5.6), 7.85 (4H, d, J=8.8), 7.34 (4H, d, J=9.2), 4.31 (4H, t, J=4.4), 3.93 (4H, t, J=4.4), 3.74 (4H, t, J=4.4), 3.64 (4H, t, J=6.0), 3.58 (4H, t, J=7.2), 1.22 (6H, t, J=6.8). δC (CD₃OD, 100 MHz, ppm): 163.19, 151.31, 146.83, 136.98, 128.26, 126.90, 122.76, 119.58, 117.45, 71.83, 70.94, 70.60, 69.50, 67.63, 15.46. δF (CD₃OD, 376 MHz, ppm): −80.44. Anal. Calc for C₃₈H₄₂F₁₂N₄O₁₄S₄ (1135.00): C, 40.21; H, 3.73; N, 4.94; 11.30%. Found C, 40.43; H, 3.71; N, 5.11; S, 11.12%.

Data for 3: Yield 89%. FTIR (NaCl, νₘₐₓ cm⁻¹): 3117, 3071, 2878, 1636, 1597, 1504, 1450, 1350, 1258, 1188, 1134, 1057, 949, 833, 787, 741, 648, 610, 571, 501. δH (CD₃OD, 400 MHz, ppm): 9.47 (4H, d, J=6.4), 8.82 (4H, d, J=6.8), 7.85 (4H, d, J=8.8), 7.33 (4H, d, J=9.2), 4.30 (4H, t, J=4.4), 3.93 (4H, t, J=4.4), 3.75 (4H, t, J=4.0), 3.51-3.69 (16H, m), 1.20 (6H, t, J=7.2). δC (CD₃OD, 100 MHz, ppm): 163.22, 151.33, 146.86, 137.00, 128.27, 126.92, 122.80, 119.61, 117.48, 71.85, 71.65, 70.95, 70.64, 69.55, 67.61, 15.48. δF (CD₃OD, 376 MHz, ppm): −80.41. Anal. Calc for C₄₂H₅₀F₁₂N₄O₁₆S₄ (1223.10): C, 41.24; H, 4.12; N, 4.58; S, 10.48%. Found C, 41.50; H, 4.21; N, 4.65; S, 10.22%.

### Example 2

Dicationic stilbazolium salts (see the structure in FIG. 1B) reached direct current conductivities in the σ_{dc}˜104.5 S·cm⁻¹ range, well above room temperature (T>80° C.) and activated by the larger free volume available beyond their glass transitions, T_{g}s. On the other hand, maximum values of σ_{dc}˜10^{2.5} S·cm⁻¹ were observed for asymmetric viologen bistriflimide salts (refer to FIG. 1C) associated with the formation of liquid crystalline smectic-T phases and correlated to short-range motions around the rod-like aromatic units. It seems that the (close) location of the N⁺ sites, and their capability to form π-π aggregates may benefit ionic conductivity. These results have prompted investigation of new triflimide viologens with extended core structures, and the conducting properties of new viologens, 1-3, prepared via Zincke reactions were investigated (refer to FIG. 1D).

Viologens and their multitude of derivatives have been postulated as functional materials in electrochromic devices, diodes and transistors, memory devices, molecular machines, and dye-sensitized solar cells. The reason to introduce the oxyethylene(s) terminations is two-fold. On the one hand, the goal was to offset (at least partially) the rigidity of the four-ring phenyl core (which could increase viscosity). Besides, the presence of polar chains can help delocalize the triflimide anions and avoid complexation, which would ultimately inhibit ion mobility.

The 4-oligoethyleneoxypheylanilines were prepared according to modified literature procedures. The synthesis of bis-(4-oligoethyleneoxyphenyl)-4,4′-bipyridinium dichlorides (P1-P3) with different ethyleneoxy groups, is summarized in Scheme 1. The method involved: (i) the aromatic nucleophilic substitution between the 1-chloro-2,4-dinitrobenzene and 4,4′-bipyridine in acetonitrile under reflux, to yield the so-called Zincke salts (Steps 1-2); and (ii) subsequent anionic ring opening and ring closing reactions (ANROC) with the corresponding 4-oligoethyleneoxypheylanilines, in N,N-dimethylacetamide (DMAc) at room temperature (Steps 3-4). Lastly, P1-P3 were converted to the 1-3 salts understudy by metathesis with lithium triflimides in methanol (Step 5). Each of the prepared salts was in brown powdered form. The chemical structures of the intermediates and final products were confirmed by Fourier transform infrared (FT-IR) spectroscopy, ¹H, ¹³C, and ¹⁹F nuclear magnetic resonance (NMR) obtained in CD₃OD, and their purities were determined by elemental analysis. These appear to be the first examples of ionic liquids prepared via Zincke reactions.

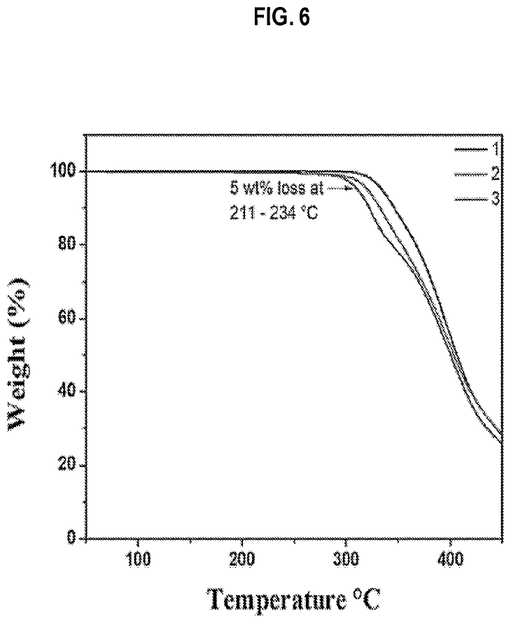

The thermal properties and phase behavior of the new salts were determined by thermogravimetric analysis (TGA), differential scanning calorimetry (DSC) and polarized optical microscopy (POM). The three salts display less than 5% weight loss up to 300° C. (degradation temperatures, T_{d}˜311-334° C.) under nitrogen atmosphere (FIG. 6, Table 1). While it was thought that the bistriflimide ions may confer high thermal stabilities, the high T_{d} values confirm that the presence of flexible oxyethylene groups do not have a destabilizing effect on the salts.

TABLE 1
Thermal Properties of Salts 1-3 as determined by DSC and TGA

| Salt | Tₘ^{a} (° C.) | T_{d}^{b} (° C.) |
|---|---|---|
| 1 | 115 (13.84) | 334 |
| 2 | 72 (3.03)^{c}, 91 (8.52) | 321 |
| 3 | −6^{d} | 311 |

^{a}Tₘ = The melting temperature was the temperature it transformed from crystal-to-liquid transition, taken from the first heating cycle of DSC thermogram at a heating rate of 10° C./min, since there was no endotherm in the subsequent second heating cycle. Enthalpy of transition in kJ · mol⁻¹ was given in parentheses.
^{b}T_{d} = the temperature at which a 5% weight loss of the salt occurred at heating rate of 10° C./min in nitrogen.
^{c}Crystal-to-crystal transition that was verified on observation of birefringent texture including defects and absence of homogeneity.
^{d}T_{g} = glass transition temperature.

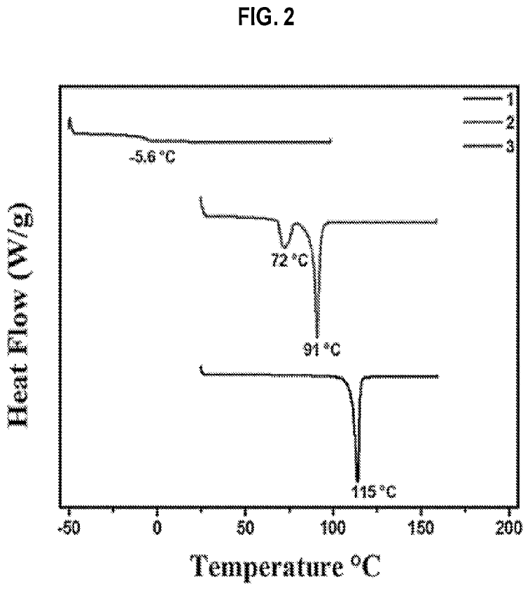

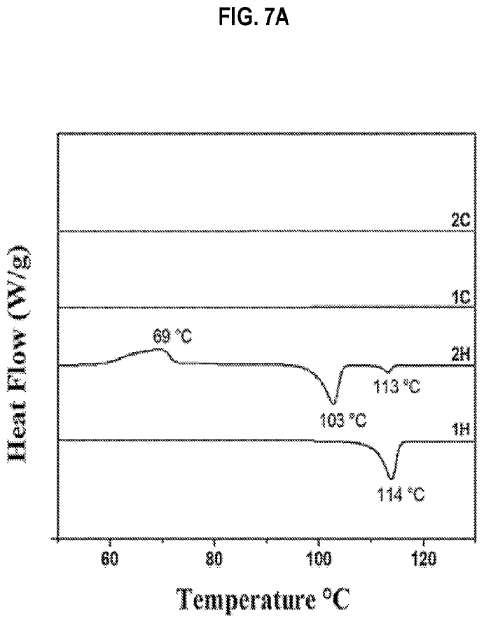

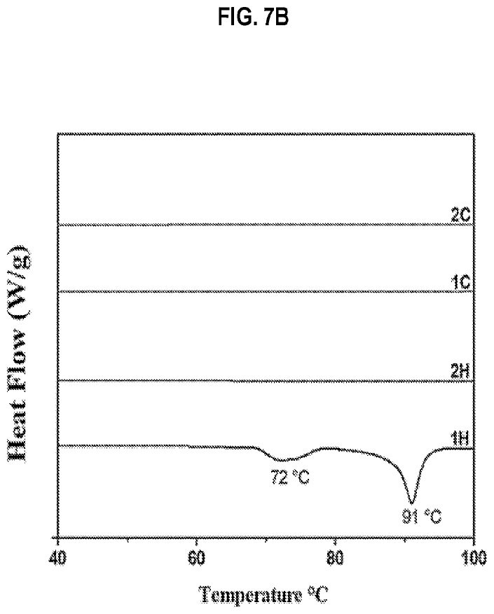

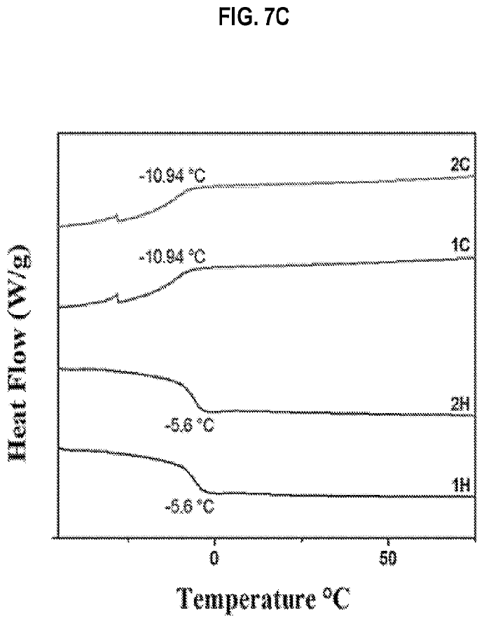

FIG. 2 shows the DSC thermograms of the three salts, corresponding to their first heating scans obtained at 10° C.·min⁻¹ rates. While 1 and 2 display first-order endotherms associated to crystal to crystal (2) and melting (1 and 2) processes, 3 only displays a glass transition at low temperature (T_{g}=−6° C. According to these results, both 1 and 2 act as ionic liquids that melt on heating (an increase in the oxyethylene termination length reduces the melting point). The absence of first-order transitions in the corresponding thermogram indicates that 3 behaves like an amorphous salt, due to inhibition of crystallization at sufficiently long ethyleneoxy chains, n=3. It is worth noting that there are no further thermal events visible in subsequent heating and cooling scans of 1 and 2, suggesting that crystallization of these samples must be a slow process, see FIG. 7A-C. The absence of liquid crystal behavior contrasts with the formation of smectic phases by analogous alkoxy-terminated (n 6) viologens observed previously by this research. Comparable lengths of terminal chains (refer to FIG. 1E) were thought to promote microphase separation and smectic behavior in 1-3, the formation of stronger interactions by the ethyleneoxy groups may restrict the local mobility required to yield liquid crystallinity. The effect of the terminal chain lengths on nanosegregation between the polar chains and the aromatic cores in similar viologens is the object of further ongoing research.

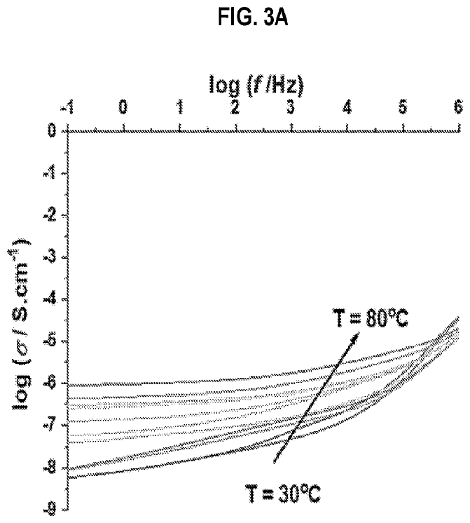

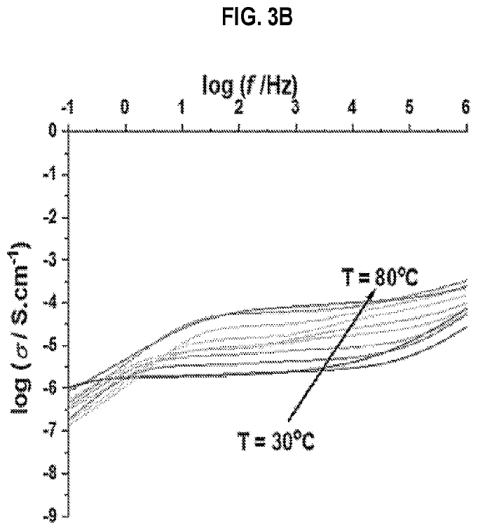

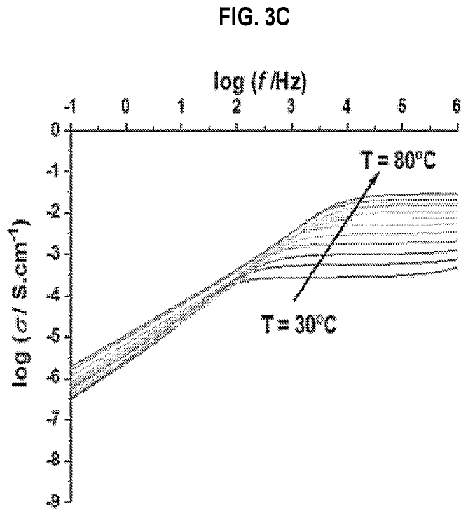

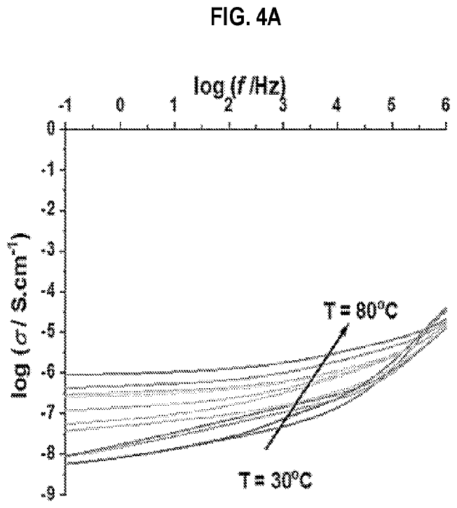

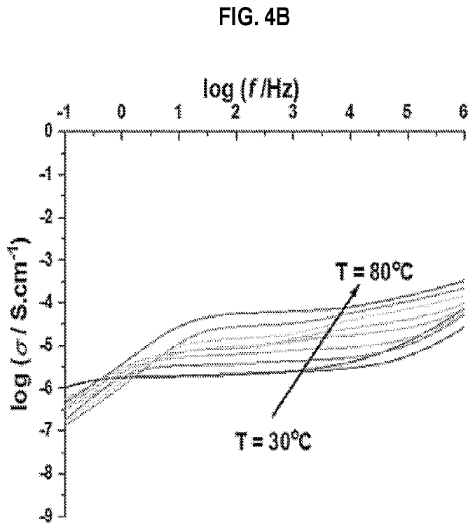

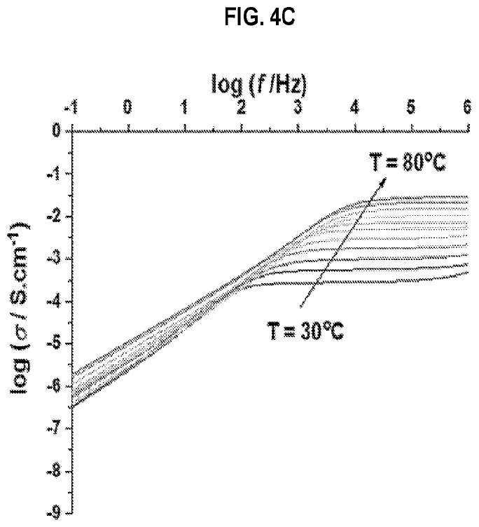

The conductivity response of the viologens is illustrated in FIG. 3 which shows their dielectric loss factor, ε″, obtained as a function of the temperature and frequency, and measured in isothermal steps on heating from room temperature, see the supplementary information for further details. The values are remarkably high for organic media, which is attributed to the strong polar character of ionic liquids and salts. All double logarithmic ε″ plots show linear drops (with slopes˜−1) that denote the rise of direct current (DC) conductivity at sufficiently low frequencies. This DC component overshadows any potential dielectric relaxation, even though some peaks are observed for 2 and 3 in FIGS. 3B and 3C, respectively. For these ionic liquids, the isotherms shift with temperature, denoting thermal activation effects that will be reviewed later. The salt 3, on the other hand, depicts the highest ε″ values among the three salts, see FIG. 3C. The occurrence of direct current conductivity is confirmed by the formation of plateaus in the double logarithmic σ′ vs f plots in FIG. 4, which have similar temperature dependences as the corresponding dielectric loss moduli depicted in FIG. 3. The DC conductivity values, σ_{dc}, can be estimated by extrapolating the constant α′ ranges to f→0 at each temperature, and the resulting Arrhenius plots are shown in FIG. 5. The activation energies of the conductivity process from the Arrhenius plot, Eₐ, are calculated using the equation; σ_{dc}=₀ exp(Eₐ/RT), where R is the gas constant, 8.31 J·mol⁻¹·K⁻¹, T is the absolute temperature, and σ₀ is a pre-exponential term.

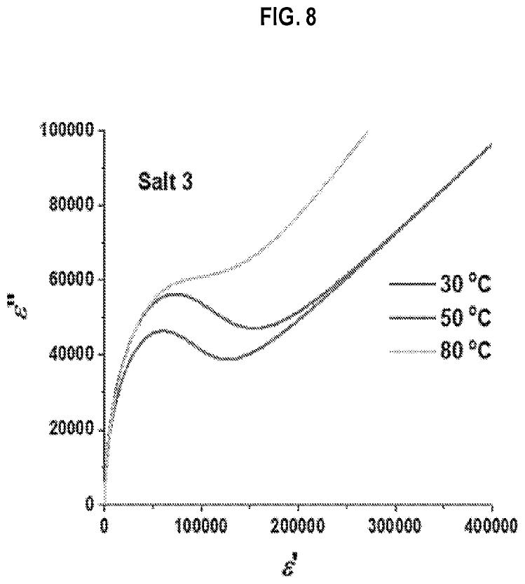

The Cole-Cole plot of 3 is shown in FIG. 8. The AC oscillation voltage was 0.5 V. The results indicate that longer ethyleneoxy terminal chains promote conductivity in the salts. Indeed, 3 shows exceptional σ_{dc} values (between 10^{-3.5} and 10^{-1.5} S·cm⁻¹) comparable to bench electrolytes used in fuel cells and batteries. This material is one of the few examples of an organic salt exhibiting such large conductivities under anhydrous conditions, and at mild temperatures, even close to room temperature. The activation energies estimated from the Arrhenius plots of the samples are Eₐ=95.9 kJ·mol⁻¹ for 1; Eₐ=84.5 kJ·mol⁻¹ for 2; and Eₐ=79.4 kJ·mol⁻¹, for 3.

These values are considerably high for locally activated processes and are in good agreement with the occurrence of so-called β-relaxations, involving the rotation of rod-like molecules (extended viologen moieties) around their long axis within the crystal lattice. It seems that, when the —(CH₂CH₂O)— terminal chains are short, the conductivity process is dominated (and partially hindered) by the motions around the bulky four-phenyl core. In salt 3, alternatively, the plasticizing effect of the longer terminal chains endows in the formation of a rubbery phase above its low glass transition (T_{g}˜−6° C., see FIG. 2), with large free volumes that facilitate ionic motion (resulting in high σ_{dc} values and slightly lower activation energy).

New viologens using Zincke reactions have been prepared, resulting in ionic liquids and salts with strong dielectric responses, attributed to the presence of both flexible oxyethylene groups and triflimide ions. Sufficiently long terminal chains promote exceptionally high ionic conductivities at room temperature, comparable to benchmark electrolytes used commercially, highlighting their potential use in energy devices, such as, fuel cells, batteries, supercapacitors, or solar cells. This work opens new horizons for designing ionic liquids with tuned electrostatic interactions and nanostructures by extending the central rigid core, exchanging different cations, or modifying the length of the oxyethylene terminations.

What is claimed:

1. An ionic liquid electrolyte comprising:
a viologen salt of formula (I): 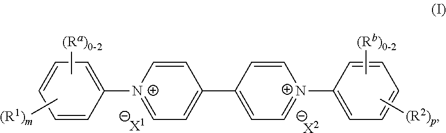
wherein:
R¹ and R² are each independently 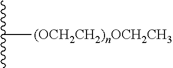
where n is 3-10;
m and p are each independently 1-3;
R^{a}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃;
R^{b}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃; and
^{⊖}X¹ and ^{⊖}X² are each 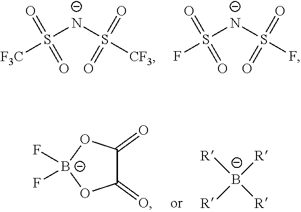
where R′ is —(OCH₂CH₂)₁₋₁₀OCH₂CH₃.

2. The ionic liquid electrolyte of claim 1, wherein m is 1.

3. The ionic liquid electrolyte of claim 1, wherein p is 1.

4. The ionic liquid electrolyte of claim 1, wherein m is 2.

5. The ionic liquid electrolyte of claim 1, wherein p is 2.

6. The ionic liquid electrolyte of claim 1, wherein the viologen salt of formula (I) is a symmetric viologen salt.

7. The ionic liquid electrolyte of claim 1, wherein ^{⊖}X¹ and ^{⊖}X² are each

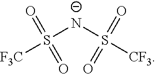 [SMILES: O=S(=O)([N-]S(=O)(=O)C(F)(F)F)C(F)(F)F]

8. The ionic liquid electrolyte of claim 1, wherein the viologen salt of formula (I) is a viologen salt of formula (I-a):

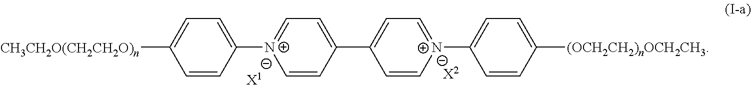

9. The ionic liquid electrolyte of claim 1, wherein the viologen salt of formula (I) is a viologen salt of formula (I-b):

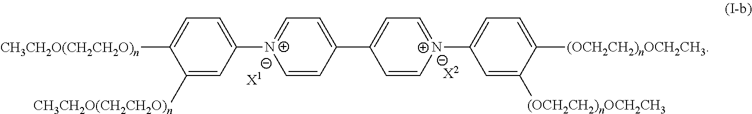

10. The ionic liquid electrolyte of claim 1, wherein the viologen salt of formula (I) is:

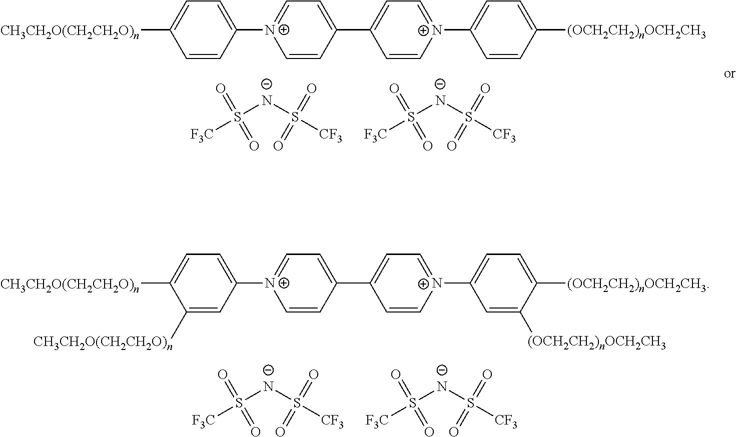

11. A solid-state battery, supercapacitor, solar cell, or perovskite photovoltaic cell comprising the ionic liquid electrolyte of claim 1.

12. The ionic liquid electrolyte of claim 1, wherein the viologen salt of formula (I) has a conductivity of greater than 0.1 S·cm⁻¹.

13. A solid-state battery comprising
a cathode;
an anode;
a separator; and
an ionic liquid electrolyte, wherein the ionic liquid electrolyte comprises:
a viologen salt of formula (I): 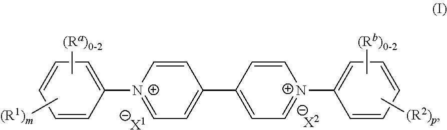
wherein:
R¹ and R² are each independently 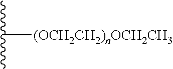
where n is 3-10;
m and p are each independently 1-3;
R^{a}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃;
R^{b}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃; and
^{⊖}X¹ and ^{⊖}X² are each 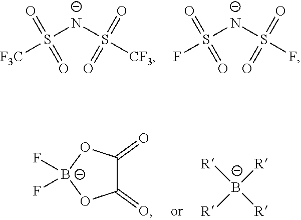
where R′ is —(OCH₂CH₂)₁₋₁₀OCH₂CH₃.

14. A supercapacitor comprising:
a cathode;
an anode;
a separator; and
an ionic liquid electrolyte, wherein the ionic liquid electrolyte comprises:
a viologen salt of formula (I): 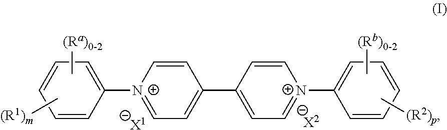
wherein:
R¹ and R² are each independently 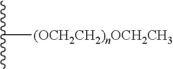
where n is 3-10;
m and p are each independently 1-3;
R^{a}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃;
R^{b}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃; and
^{⊖}X¹ and ^{⊖}X² are each 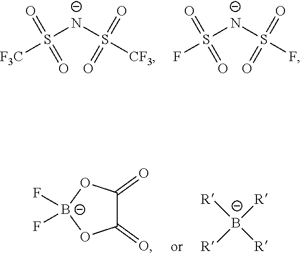
where R′ is —(OCH₂CH₂)₁₋₁₀OCH₂CH₃.

15. A solar cell comprising:
a n-type semiconductor layer;
a p-type semiconductor layer; and
an ionic liquid electrolyte, wherein the ionic liquid electrolyte comprises:
a viologen salt of formula (I): 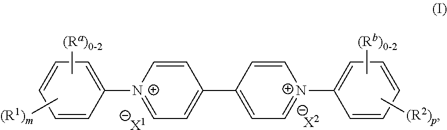
wherein:
R¹ and R² are each independently 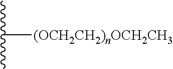
where n is 3-10;
m and p are each independently 1-3;
R^{a}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃;
R^{b}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃; and
^{⊖}X¹ and ^{⊖}X² are each 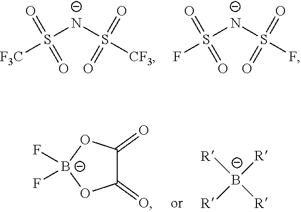
where R′ is —(OCH₂CH₂)₁₋₁₀OCH₂CH₃.

16. A perovskite photovoltaic cell comprising:
a n-type semiconductor layer;
an electron transport layer (ETL);
a p-type semiconductor layer; and
an ionic liquid electrolyte, wherein the ionic liquid electrolyte comprises:
a viologen salt of formula (I): 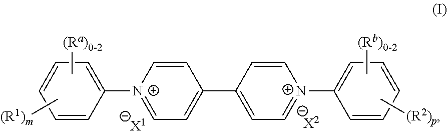
wherein:
R¹ and R² are each independently 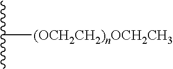
where n is 3-10;
m and p are each independently 1-3;
R^{a}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃;
R^{b}, at each occurrence, is independently C₁₋₄alkyl, C₁₋₂haloalkyl, —OC₁₋₄alkyl, —OC₁₋₂haloalkyl, halogen, —NO₂, —CN, or —(OCH₂CH₂)₁₋₃OCH₂CH₃; and
^{⊖}X¹ and ^{⊖}X² are each 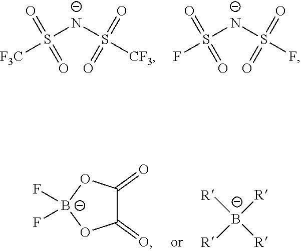
where R′ is —(OCH₂CH₂)₁₋₁₀OCH₂CH₃.

17. The solid-state battery, supercapacitor, solar cell, or perovskite photovoltaic cell of any one of claims 13-16, wherein m is 1.

18. The solid-state battery, supercapacitor, solar cell, or perovskite photovoltaic cell of any one of claims 13-16, wherein p is 1.

19. The solid-state battery, supercapacitor, solar cell, or perovskite photovoltaic cell of any one of claims 13-16, wherein m is 2.

20. The solid-state battery, supercapacitor, solar cell, or perovskite photovoltaic cell of any one of claims 13-16, wherein p is 2.

21. The solid-state battery, supercapacitor, solar cell, or perovskite photovoltaic cell of any one of claims 13-16, wherein the viologen salt is a symmetric viologen salt.

22. The solid-state battery, supercapacitor, solar cell, or perovskite photovoltaic cell of any one of claims 13-16, wherein ^{⊖}X¹ and ^{⊖}X² are each

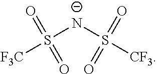 [SMILES: O=S(=O)([N-]S(=O)(=O)C(F)(F)F)C(F)(F)F]

23. The solid-state battery, supercapacitor, solar cell, or perovskite photovoltaic cell of any one of claims 13-16, wherein the viologen salt of formula (I) is a viologen salt of formula (I-a):

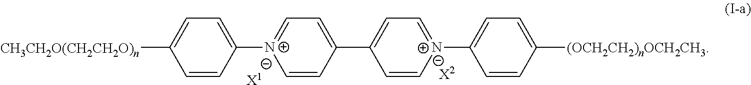

24. The solid-state battery, supercapacitor, solar cell, or perovskite photovoltaic cell of any one of claims 13-16, wherein the viologen salt of formula (I) is a viologen salt of formula (I-b):

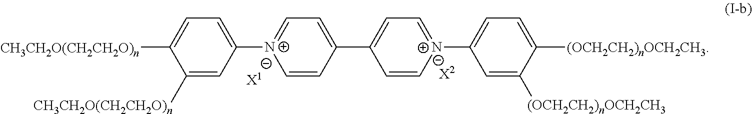

25. The solid-state battery, supercapacitor, solar cell, or perovskite photovoltaic cell of any one of claims 13-16, wherein the viologen salt of formula (I) has a conductivity of greater than 0.1 S·cm⁻¹.
